(19) United States
(12) Patent Application Publication
(10) Pub. No.: US 2006/0134534 A1
(43) Pub. Date: Jun. 22, 2006

(54) Photomask and method for maintaining optical properties of the same

(76) Inventors: Laurent Dieu, Austin, TX (US); Joseph Stephen Gordon, Gardiner, NY (US); Eric Vincent Johnstone, Round Rock, TX (US); Christian Chovino, Round Rock, TX (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 11/349,438

(22) Filed: Feb. 7, 2006

Related U.S. Application Data
(63) Continuation of application No. PCT/US04/27435, filed on Aug. 24, 2004.
(60) Provisional application No. 60/497,541, filed on Aug. 25, 2003.

(51) Int. Cl. B08B 7/00; B08B 6/00; G21G 5/00; G03C 5/00; G03F 1/00; B08B 3/12; B08B 7/02; A61N 5/00

(52) U.S. Cl. 430/5; 250/492.3; 134/1; 430/322

(57) ABSTRACT

A photomask and method for maintaining optical properties of the same are disclosed. The method includes providing a substrate including a first surface having an absorber layer formed thereon and a second surface located opposite the first surface. A pattern is formed in the absorber layer to create a photomask for use in a semiconductor manufacturing process. A transmissive protective layer is also formed on at least one of the patterned layer and the second surface of the substrate. The protective layer reduces haze growth when the photomask is used in the semiconductor manufacturing process.

### RELATED APPLICATION

This application is a Continuation of International Patent Application No. PCT/US04/27435 filed Aug. 24, 2004, which designates the United States and claims the benefit of U.S. Provisional Patent Application Ser. No. 60/497,541, entitled “Photomask and Method for Maintaining Optical Properties of the Same” filed by Laurent Dieu et al. on Aug. 25, 2003, which are incorporated herein by reference in their entirety.

### TECHNICAL FIELD OF THE INVENTION

The present invention relates in general to semiconductor device manufacturing and, more particularly to a photomask and method for maintaining optical properties of the same.

### BACKGROUND OF THE INVENTION

As semiconductor device manufacturers continue to produce smaller devices, the requirements for photomasks used in the fabrication of these devices continue to tighten. Photomasks, also known as reticles or masks, typically consist of substrates (e.g., high-purity quartz or glass) that have an opaque and/or partially opaque layer (e.g., chrome) formed on the substrate. The opaque layer includes a pattern representing a circuit image that may be transferred onto semiconductor wafers in a lithography system. As feature sizes of semiconductor devices decrease, the corresponding circuit images on the photomask also become smaller and more complex. Consequently, the quality of the mask has become one of the most crucial elements in establishing a robust and reliable semiconductor fabrication process.

Characteristics of the photomask that define its quality include the flatness of the substrate, the dimensions of the features formed by the opaque layer and the transmission properties of the substrate and the absorber layer. These characteristics may be altered by various procedures during the manufacturing process, which may reduce the quality of the photomask. For example, the photomask is typically cleaned at least one time during the manufacturing process to remove any contaminants that may be present on the exposed surfaces. The cleaning process, however, can leave a chemical residue on the exposed surfaces. This residue may react with contaminants that may be created by a lithography system and cause a haze to grow on the exposed surfaces, which may alter the transmission properties of the photomask. If the transmission properties of the photomask are altered, the pattern from the photomask may not be accurately transferred to a semiconductor wafer, thus causing defects or errors in the microelectronic devices formed on the wafer.

Traditionally, the haze may be removed by wiping the surface of the substrate after the photomask is used numerous times in a semiconductor manufacturing process. However, wiping the surface of the substrate may create scratches and/or add other types of contaminants on the surface. These additional contaminants and scratches may further degrade the quality of the photomask. Furthermore, wiping the surface may not prevent a haze from forming on the photomask when used in the semiconductor manufacturing process again.

### SUMMARY OF THE INVENTION

In accordance with teachings of the present invention, disadvantages and problems associated with maintaining optical properties of a photomask have been substantially reduced or eliminated. In a particular embodiment, a protective layer is formed on a bottom surface of a substrate that prevents a haze from growing on the bottom surface during a lithography process.

In accordance with one embodiment of the present invention, a method for maintaining optical properties of a photomask includes providing a substrate including a first surface having an absorber layer formed thereon and a second surface located opposite the first surface. A pattern is formed in the absorber layer to create a photomask for use in a semiconductor manufacturing process. A transmissive protective layer is also formed on at least one of the patterned layer and the second surface of the substrate. The protective layer prevents haze growth when the photomask is used in the semiconductor manufacturing process.

In accordance with another embodiment of the present invention, a method for maintaining optical properties of a photomask includes providing a photomask blank. The photomask blank includes a substrate having a first surface and a second surface located opposite the first surface. The first surface includes an absorber layer formed on at least a portion thereof. A protective layer is formed on at least a portion of the second surface of the substrate. The protective layer prevents haze growth on a photomask fabricated from the photomask blank when the photomask is used in a semiconductor manufacturing process.

In accordance with a further embodiment of the present invention, a photomask includes a patterned layer formed on at least a portion of a first surface of a substrate and a protective layer formed on at least one of the patterned layer and a second surface of the substrate. The protective layer prevents haze growth when the photomask is used in a semiconductor manufacturing process.

Important technical advantages of certain embodiments of the present invention include a protective layer that prevents optical properties associated with a photomask from degrading. The protective layer may be formed on an exposed surface of the substrate at the beginning of a photomask manufacturing process. During the manufacturing process, the photomask may be cleaned and a chemical residue from the cleaning solution may form on the protective layer. By removing the protective layer after the final cleaning process, the surfaces of the photomask may be free of residue, which prevents the formation of haze caused by the reaction between the residue and contaminants in a lithography system. The optical properties of the photomask, therefore, are maintained after multiple uses in a semiconductor manufacturing process.

Another important technical advantage of certain embodiments of the present invention includes a protective layer that improves optical properties associated with a photomask. During a photomask manufacturing process, the photomask may be cleaned multiple times and each cleaning process may leave chemical residue on exposed surfaces of the substrate. The protective layer may be formed on an exposed surface of the substrate after the final cleaning process to act as a coating that covers any residue left by the cleaning solution. The protective layer may further have a thickness tuned to produce a transmission maximum at an exposure wavelength of a lithography system, which may enhance the optical properties of the photomask.

All, some, or none of these technical advantages may be present in various embodiments of the present invention. Other technical advantages will be readily apparent to one skilled in the art from the following figures, descriptions, and claims.

### BRIEF DESCRIPTION OF THE DRAWINGS

A more complete and thorough understanding of the present embodiments and advantages thereof may be acquired by referring to the following description taken in conjunction with the accompanying drawings, in which like reference numbers indicate like features, and wherein:

FIG. 1 illustrates a cross-sectional view of a photomask assembly that includes a protective layer formed on a surface of a substrate according to teachings of the present invention;

FIG. 2 illustrates a cross sectional view of a photomask blank that includes a protective layer formed on a surface of a substrate according to teachings of the present invention; and

FIG. 3 illustrates a flow chart of a method for maintaining optical properties of a photomask according to teachings of the present invention.

### DETAILED DESCRIPTION OF THE INVENTION

Preferred embodiments of the present invention and their advantages are best understood by reference to FIGS. 1 through 3, where like numbers are used to indicate like and corresponding parts.

FIG. 1 illustrates a cross-sectional view of a photomask assembly including a protective layer formed on at least one surface of the substrate. Photomask assembly 10 includes pellicle assembly 14 mounted on photomask 12. Substrate 16 and patterned layer 18 form photomask 12, also known as a mask or reticle, that may have a variety of sizes and shapes, including but not limited to round, rectangular, or square. Photomask 12 may also be any variety of photomask types, including, but not limited to, a one-time master, a five-inch reticle, a six-inch reticle, a nine-inch reticle or any other appropriately sized reticle that may be used to project an image of a circuit pattern onto a semiconductor wafer. Photomask 12 may further be a binary mask, a phase shift mask (PSM) (e.g., an alternating aperture phase shift mask, also known as a Levenson type mask), an optical proximity correction (OPC) mask or any other type of mask suitable for use in a lithography system.

Photomask 12 includes patterned layer 18 formed on top surface 17 of substrate 16 that, when exposed to electromagnetic energy in a lithography system, projects a pattern onto a surface of a semiconductor wafer (not expressly shown). Substrate 16 may be a transparent material such as quartz, synthetic quartz, fused silica, magnesium fluoride (MgF₂), calcium fluoride (CaF₂), or any other suitable material that transmits at least seventy-five percent (75%) of incident light having a wavelength between approximately 10 nanometers (nm) and approximately 450 nm. In an alternative embodiment, substrate 16 may be a reflective material such as silicon or any other suitable material that reflects greater than approximately fifty percent (50%) of incident light having a wavelength between approximately 10 nm and 450 nm.

Patterned layer 18 may be a metal material such as chrome, chromium nitride, a metallic oxy-carbo-nitride (e.g., MOCN, where M is selected from the group consisting of chromium, cobalt, iron, zinc, molybdenum, niobium, tantalum, titanium, tungsten, aluminum, magnesium, and silicon), or any other suitable material that absorbs electromagnetic energy with wavelengths in the ultraviolet (UV) range, deep ultraviolet (DUV) range, vacuum ultraviolet (VUV) range and extreme ultraviolet range (EUV). In an alternative embodiment, patterned layer 18 may be a partially transmissive material, such as molybdenum silicide (MoSi), which has a transmissivity of approximately one percent (1%) to approximately thirty percent (30%) in the UV, DUV, VUV and EUV ranges.

Frame 20 and pellicle film 22 may form pellicle assembly 14. Frame 20 is typically formed of anodized aluminum, although it could alternatively be formed of stainless steel, plastic or other suitable materials that do not degrade or outgas when exposed to electromagnetic energy within a lithography system. Pellicle film 22 may be a thin film membrane formed of a material such as nitrocellulose, cellulose acetate, an amorphous fluoropolymer, such as TEFLON® AF manufactured by E. I. du Pont de Nemours and Company or CYTOP® manufactured by Asahi Glass, or another suitable film that is transparent to wavelengths in the UV, DUV, EUV and/or VUV ranges. Pellicle film 22 may be prepared by a conventional technique such as spin casting.

Pellicle film 22 protects photomask 12 from contaminants, such as dust particles, by ensuring that the contaminants remain a defined distance away from photomask 12. This may be especially important in a lithography system. During a lithography process, photomask assembly 10 is exposed to electromagnetic energy produced by a radiant energy source within the lithography system. The electromagnetic energy may include light of various wavelengths, such as wavelengths approximately between the I-line and G-line of a Mercury arc lamp, or DUV, VUV or EUV light. In operation, pellicle film 22 is designed to allow a large percentage of the electromagnetic energy to pass through it. Contaminants collected on pellicle film 22 will likely be out of focus at the surface of the wafer being processed and, therefore, the exposed image on the wafer should be clear. Pellicle film 22 formed in accordance with the teachings of the present invention may be satisfactorily used with all types of electromagnetic energy and is not limited to lightwaves as described in this application.

In the illustrated embodiment, protective layer 24 may be formed on bottom surface 19 of substrate 16 opposite patterned layer 18. In another embodiment, protective layer 24 may be formed on either or both of patterned layer 18 and bottom surface 19. Protective layer 24 may be formed of a material such as an amorphous fluoropolymer (e.g., TEFLON® AF manufactured by E.I. du Pont de Nemours and Company or CYTOP® manufactured by Asahi Glass), diamond like carbon (DLC), aluminum oxide (Al₂O₃), hafnium oxide (HfO), magnesium fluoride (MgF₂), calcium fluoride (CaF₂), or any other suitable material. In one embodiment, the material forming protective layer 24 may be substantially transparent to wavelengths in the UV, DUV, EUV and/or VUV ranges and may be tuned to improve the optical properties of photomask 12. In an alternative embodiment, protective layer 24 may be a material, such as an amorphous fluoropolymer, that absorbs at least a percentage of radiant energy having a wavelength less than or equal to approximately 450 nanometers.

Photomask 12 may be formed from a photomask blank using a standard lithography process. In a lithography process, a mask pattern file that includes data for patterned layer 18 may be generated from a mask layout file. In one embodiment, the mask layout file may include polygons that represent transistors and electrical connections for an integrated circuit. The polygons in the mask layout file may further represent different layers of the integrated circuit when it is fabricated on a semiconductor wafer. For example, a transistor may be formed on a semiconductor wafer with a diffusion layer and a polysilicon layer. The mask layout file, therefore, may include one or more polygons drawn on the diffusion layer and one or more polygons drawn on the polysilicon layer. The polygons for each layer may be converted into a mask pattern file that represents one layer of the integrated circuit. Each mask pattern file may be used to generate a photomask for the specific layer. In some embodiments, the mask pattern file may include more than one layer of the integrated circuit such that a photomask may be used to image features from more than one layer onto the surface of a semiconductor wafer.

The desired pattern may be imaged into a resist layer of the photomask blank using a laser, electron beam or X-ray lithography system. In one embodiment, a laser lithography system uses an Argon-Ion laser that emits light having a wavelength of approximately 364 nanometers (nm). In alternative embodiments, the laser lithography system uses lasers emitting light at wavelengths from approximately 150 nm to approximately 300 nm. Photomask 12 may be fabricated by developing and etching exposed areas of the resist layer to create a pattern, etching the portions of patterned layer 18 and protective layer 24, if formed on patterned layer 18, not covered by resist, and removing the undeveloped resist to create patterned layer 18 over substrate 16.

During a photomask manufacturing process, photomask 12 may be cleaned multiple times in order to remove contaminants created during the photomask manufacturing process from the surfaces of photomask 12. The cleaning solution used in the cleaning processes, however, may leave residue, including but not limited to, nitrogen acid compounds and sulfuric base compounds, on the exposed surfaces of photomask 12. In one embodiment, protective layer 24 maybe be formed on bottom surface 19 of substrate 16 by a photomask blank manufacturer or a photomask manufacturer before photomask 12 is subjected to a cleaning process. In another embodiment, protective layer 24 may additionally be formed by the photomask blank manufacturer on the absorber layer used to create patterned layer 18 on top surface 17 of substrate 16. After a final cleaning process, protective layer 24 may be removed in order to prevent any residue left by the cleaning solution from reacting with contaminants in a lithography system and forming a haze (e.g., a layer of crystallized material) on photomask 12. Protective layer 24 may act as a protective coating for photomask 12 during the photomask manufacturing process such that any residue from the cleaning solution forms on protective layer 24. Photomask 12, therefore, is free of any residue or contaminants when photomask 12 is used in a semiconductor manufacturing process. Once protective layer 24 is removed, pellicle assembly 14 may then be mounted on top surface 17 of substrate 16 to protect patterned layer 18 during the semiconductor manufacturing process.

In a further embodiment, photomask 12 may be manufactured and protective layer 24 may be formed on either or both of patterned layer 18 and bottom surface 19 of substrate 16 after a final cleaning process. As described above, photomask 12 may be cleaned multiple times during a photomask manufacturing process. The cleaning solution used in the cleaning processes may leave residue, such as nitrogen acid compounds and sulfuric base compounds, on patterned layer 18 and/or bottom surface 19 of substrate 16. Protective layer 24 may be formed on photomask 12 in order to prevent the residue formed on either or both of patterned layer 18 and bottom surface 19 of substrate 16 from reacting with contaminants in a lithography system and forming a haze on photomask 12. Since protective layer 24 was formed on photomask 12 after the final clean, the exposed surface of protective layer 24 may be free of any residue that may cause a haze to form during a semiconductor manufacturing process. Additionally, protective layer 24 may be a material including a thickness tuned to enhance the optical properties of photomask 12 during the semiconductor manufacturing process. For example, the thickness of protective layer 24 may be tuned to maximize transmission of one or more exposure wavelengths in a lithography system. Once protective layer 24 is formed, pellicle assembly 14 may then be mounted on top surface 17 of substrate 16 to protect patterned layer 18 during the semiconductor manufacturing process.

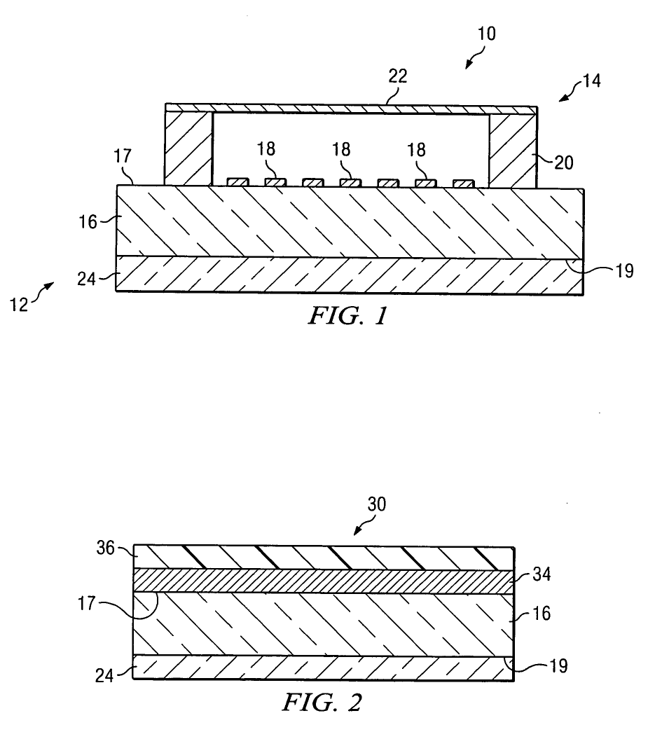

FIG. 2 illustrates a cross-sectional view of photomask blank 30 used to manufacture photomask 12. Photomask blank 30 may include substrate 16 having absorber layer 34 formed on top surface 17 of substrate 16. Patterned layer 18, as described with respect to FIG. 1, may be formed by imaging a pattern in absorber layer 34. Absorber layer 34, therefore, may include the same materials used for patterned layer 18. Although absorber layer 34 is illustrated as a single layer composed of a single type of material, absorber layer 34 may be multiple layers of different materials or a single graded layer composed of different material. Resist layer 36 may be formed on absorber layer 34. Resist layer 36 may be any suitable positive or negative resist for use in a lithography system using an exposure wavelength between approximately 150 nm to approximately 450 nm.

In the illustrated embodiment, photomask blank 30 may further include protective layer 24 formed on bottom surface 19 of substrate 16. In other embodiments, protective layer may be formed on either or both of absorber layer 34 and bottom surface 19 of substrate 16. Protective layer 24 may have a thickness that produces a transmission maximum at a specific exposure wavelength. For example, if the exposure wavelength of a lithography system is approximately 248 nm, protective layer 24 may be tuned to have a thickness that maximizes transmission of the exposure wavelength. In one embodiment, protective layer 24 may be formed on substrate 16 by a photomask blank manufacturer after resist layer 36 is formed on absorber layer 34. In another embodiment, protective layer 24 may be formed on either or both of absorber layer 34 and bottom surface 19 of substrate 16 before resist layer 36 is formed on photomask blank 30. The photomask blank manufacturer may then ship photomask blank 30 to a photomask manufacturer. In another embodiment, the photomask blank manufacturer may ship photomask blank 30 to the photomask manufacturer without protective layer 24. In this example, the photomask manufacturer may form protective layer 24 on bottom surface 19 of substrate 16 in a photomask manufacturing facility.

In either embodiment, photomask blank 30 including protective layer 24 may be used to manufacture a photomask, such as photomask 12 described above in reference to FIG. 1. After a final cleaning process is used to remove contaminants and prior to mounting a pellicle assembly on the top surface of a photomask formed from photomask blank 30, protective layer 24 may be removed in order to provide patterned layer 18 and/or bottom surface 19 of substrate 16 that is free of residue from the cleaning processes. During a photomask manufacturing process, the photomask may be cleaned multiple times. The cleaning solution used may leave residue, such as nitrogen acid compounds and sulfuric base compounds, on patterned layer 18 and/or bottom surfaces 19 of the photomask. During a semiconductor manufacturing process, the residue on the surfaces may provide nucleation sites when subjected to an exposure wavelength and the residue may react with other contaminants to form a haze on the surfaces of the photomask. This haze may degrade the optical properties of the photomask. By placing protective layer 24 on photomask 12, any residue forms on protective layer 24. The optical properties of the photomask manufactured from photomask blank 30, therefore, may be improved because haze growth will not occur during the semiconductor manufacturing process.

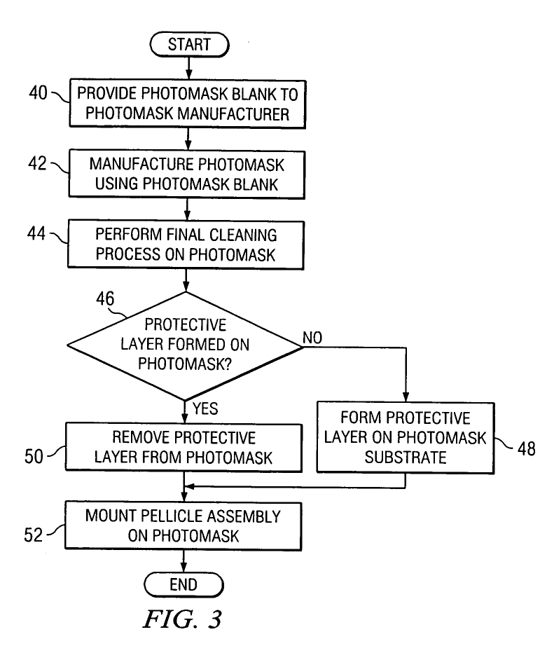

FIG. 3 illustrates a flow chart of a method for maintaining optical properties of a photomask. Generally, a protective layer may be formed on at least one surface of a photomask during a photomask manufacturing process. The protective layer prevents a haze from forming on the photomask during a semiconductor manufacturing process and simultaneously maintains and even improves optical properties associated with the photomask.

At step 40, photomask blank 30 may be provided to a photomask manufacturer. Photomask blank 30 may be manufactured using any known technique. As described above in reference to FIG. 2, photomask blank 30 may include substrate 16, absorber layer 34 and resist layer 36. In one embodiment, photomask blank 30 may additionally include protective layer 24 formed on one or both of absorber layer 34 and bottom surface 19 of substrate 16. Photomask blank 30 may be used to manufacture photomask 12 at step 42. As described above in reference to FIG. 1, any known technique may be used to form patterned layer 18 of photomask 12.

At step 44, a final cleaning process may be performed on photomask 12. In one embodiment, the cleaning solution may be an alkali solution, such as ammonia/hydrogen peroxide. In another embodiment, the cleaning solution may be a sulfuric solution. In other embodiments, the cleaning solution may be any suitable solution used to remove contaminants from the surfaces of photomask 12 without significantly affecting the optical properties of photomask 12. During the final cleaning process and any intermediate cleaning processes, residuals from the cleaning solution may form on exposed surfaces of photomask 12. These residuals may act as nucleation sites during a semiconductor manufacturing process and cause a haze to grow on the exposed surfaces. The haze may degrade the optical properties of photomask 12 causing the percentage of the exposure wavelength transmitted through substrate 16 to decrease. This decrease in transmission may effect the circuit image projected on to a semiconductor wafer and even create defects in the image.

At step 46, a photomask manufacturer may determine if protective layer 24 is formed on photomask 12. If protective layer 24 is not formed on photomask 12, protective layer 24 may be deposited on either or both of patterned layer 18 and bottom surface 19 of substrate 16 by any conventional method for forming a thin layer of material on a substrate at step 48. Protective layer may be a material such as an amorphous fluoropolymer (e.g., TEFLON® AF manufactured by E. I. du Pont de Nemours and Company or CYTOP® manufactured by Asahi Glass), diamond like carbon (DLC), aluminum oxide (Al₂O₃), hafnium oxide (HfO), magnesium fluoride (MgF₂), calcium fluoride (CaF₂), or another suitable material. In one embodiment, protective layer 24 may have a thickness tuned to maximize transmission of an exposure wavelength in a lithography system.

If the photomask manufacturer determines that protective layer 24 was formed on photomask 12 at the beginning of the photomask manufacturing process (e.g., either by the photomask manufacturer or the photomask blank manufacturer), protective layer 24 may removed at step 50. As described above, photomask 12 may be cleaned multiple times during the photomask manufacturing process and residuals from the cleaning solution may form on the exposed surfaces of photomask 12. Since protective layer 24 was formed on photomask 12 before an initial cleaning process was performed on photomask 12, any residue created by the cleaning processes may form on protective layer 24. In order to prevent reactions from occurring during a semiconductor manufacturing process, protective layer 24 may be removed from photomask 12. Photomask 12, therefore, is free of any residue since protective layer 24 was present during the cleaning processes.

At step 52, pellicle assembly 14 is mounted on photomask 12. In one embodiment, photomask 12 may include protective layer 24. In another embodiment, photomask 12 may not include any type of protective coating. In either embodiment, protective layer 24 effectively prevents a haze from forming on photomask 12 during a semiconductor manufacturing process and may even improve the optical properties of photomask 12 over its lifetime.

Although the present invention has been described with respect to a specific preferred embodiment thereof, various changes and modifications may be suggested to one skilled in the art and it is intended that the present invention encompass such changes and modifications fall within the scope of the appended claims.

What is claimed is:

1. A method for maintaining optical properties of a photomask, comprising:
providing a substrate including a first surface having an absorber layer formed thereon and a second surface located opposite the first surface;
forming a pattern in the absorber layer to create a photomask operable to be used in a semiconductor manufacturing process; and
forming a transmissive protective layer on at least one of the patterned layer and the second surface of the substrate, the protective layer operable to prevent haze growth when the photomask is used in the semiconductor manufacturing process.

2. The method of claim 1, further comprising the protective layer selected from a material consisting of an amorphous fluoropolymer, Al₂O₃, HfO, MgF₂, CaF₂ and DLC.

3. The method of claim 1, further comprising the protective layer operable to transmit radiant energy including a wavelength of less than or equal to approximately 450 nanometers.

4. The method of claim 1, further comprising the protective layer operable to provide a third surface free of residuals deposited by a cleaning solution.

5. The method of claim 1, further comprising coupling a pellicle assembly to the first surface of the substrate after forming the protective layer.

6. The method of claim 1, further comprising cleaning the photomask before forming the protective layer.

7. The method of claim 1, further comprising the protective layer including a thickness operable to provide a transmission maximum for at least one exposure wavelength associated with the semiconductor manufacturing process.

8. The method of claim 1, further comprising forming the protective on at least a portion of the second surface of the substrate.

9. The method of claim 1, further comprising forming the protective layer on at least a portion of the patterned layer.

10. A method for maintaining optical properties of a photomask, comprising:
providing a photomask blank including:
a substrate having a first surface and a second surface located opposite the first surface; and
an absorber layer formed on at least a portion of the first surface of the substrate; and
forming a first protective layer on the second surface of the substrate, the protective layer operable to prevent haze growth on a photomask fabricated from the photomask blank when the photomask is used in a semiconductor manufacturing process.

11. The method of claim 10, further comprising the photomask blank including a second protective layer formed on at least a portion of the absorber layer.

12. The method of claim 11, further comprising:
forming a pattern in the absorber layer to create the photomask operable to be used in the semiconductor manufacturing process;
cleaning the photomask after forming the pattern in the absorber layer; and
removing the first and second protective layers after cleaning the photomask such that the photomask is free of residuals created by a cleaning solution used in the cleaning process.

13. The method of claim 12, further comprising coupling a pellicle assembly to the first surface of the substrate after removing the protective layers.

14. The method of claim 10, further comprising the protective layer selected from a material consisting of an amorphous fluoropolymer, Al₂O₃, HfO, MgF₂, CaF₂ and DLC.

15. The method of claim 10, further comprising the protective layer operable to transmit radiant energy including a wavelength of less than or equal to approximately 450 nanometers.

16. The method of claim 10, further comprising the protective layer operable to absorb at least a percentage of radiant energy including a wavelength of less than or equal to approximately 450 nanometers.

17. The method of claim 10, further comprising:
forming a pattern in the absorber layer to create the photomask operable to be used in the semiconductor manufacturing process;
cleaning the photomask after forming the pattern in the absorber layer; and
removing the protective layer after cleaning the photomask such that the second surface of the substrate is free of residuals created by a cleaning solution used in the cleaning process.

18. The method of claim 17, further comprising coupling a pellicle assembly to the first surface of the substrate after removing the protective layer.

19. A photomask, comprising:
a patterned layer formed on at least a portion of a first surface of a substrate including a second surface opposite the first surface; and
a protective layer formed on at least one of the patterned layer and second surface of the substrate, the protective layer operable to prevent haze growth when the photomask is used in a semiconductor manufacturing process.

20. The photomask of claim 19, further comprising the protective layer selected from a material consisting of an amorphous fluoropolymer, Al₂O₃, HfO, MgF₂, CaF₂ and DLC.

21. The photomask of claim 19, further comprising the protective layer operable to substantially transmit radiant energy including a wavelength of less than or equal to approximately 450 nanometers.

22. The photomask of claim 19, further comprising the protective layer operable to provide a third surface free of residuals created by a cleaning solution.

23. The photomask of claim 19, further comprising a pellicle assembly coupled to the first surface of the substrate.

24. The photomask of claim 19, further comprising the protective layer including a thickness operable to provide a transmission maximum at an exposure wavelength for the semiconductor manufacturing process.
